(19) United States
(12) Patent Application Publication
(10) Pub. No.: US 2004/0115244 A1
(43) Pub. Date: Jun. 17, 2004

(54) Methods and compositions for nicotine replacement therapy

(76) Inventor: Eric Jamison Holgate, North Augusta, SC (US)

Correspondence Address: Eric Jamison Holgate, 611 Bramble Road, North Augusta, SC, 29860, US

(21) Appl. No.: 10/321,106

(22) Filed: Dec. 17, 2002

(51) Int. Cl. A61K 31/555; A61K 9/68

(52) U.S. Cl. 424/440; 514/184; 514/343

(57) ABSTRACT

Disclosed is an improved composition and method for smoking cessation. The composition includes a candy base, sweetener, nicotine, (to satisfy nicotine cravings experienced by someone attempting to discontinue smoking), and chromium picolinate, (to address the issue of undesirable weight gain associated with smoking cessation.) The method includes selective substitution of the lollipop with the cigarette until smoking ceases.

### FIELD OF THE INVENTION

This invention relates to a composition and method for treating a condition responsive to nicotine therapy, particularly smoking cessation. More particularly, this invention relates to the administration of nicotine through mucous membranes to aid in overcoming symptoms of withdrawal.

### BACKGROUND OF THE INVENTION

The health hazards of tobacco smoking are well established. Tobacco is the single greatest cause of disease and premature death in America, causing more than 430,000 deaths each year. Nearly 25% of adult Americans currently smoke, and 3,000 children and adolescents a day become regular users. Nationwide, medical care costs attributable to smoking or smoking-related diseases have been estimated by the Centers for Disease Control to be more than $50 billion annually.

More than 80% of all smokers express a desire to stop smoking, and each year 35% try to stop smoking, but less than 5% are successful in unaided attempts. Smokers, on average, attempt to quit five times before succeeding.

Nicotine addiction results in withdrawal symptoms in an individual attempting smoking cessation. The severity of the symptoms depends on the individual and the length of time in using tobacco products. This withdrawal often leads to symptoms such as lack of concentration, anxiety, depression, anger, restlessness, frustration, insomnia, irritation, and increased appetite. It has also been demonstrated that abrupt cessation of nicotine consumption can deaden the brain's sensitivity to positive stimulus.

Several commercially available products have been introduced to ease smoking cessation. Nicotine replacement therapy has become one of the most common aids to quitting smoking. Currently, there are nicotine lozenges, nicotine gum and trans-dermal patches on the market.

However, these products all administer fixed doses of nicotine to an individual. Nicotine, as with most drugs, has different effects on different people. These effects depend upon the individual's tobacco consumption habits, the strength of their normal product, genetic differences, blood volumes, and diet, among numerous other factors. The administration of set doses of nicotine therefore provides different results and may actually decrease the likelihood of success through either overdosing or underdosing any given individual.

In addition, it has become increasingly clear that simple replacement does not adequately address the symptoms associated with the cessation of smoking. The products currently on the market do not address the psychological factors that also seem to play a role, with smoking being associated with certain places, events or activities. One such psychological factor is the habit of holding something in the hands, as well as the hand-to-mouth stimulation associated with cigarette smoking. Further, certain individuals begin to eat compulsively following smoking cessation. The increased appetite and compulsive eating may lead to weight gain unless the individuals alter their eating habits along with ceasing smoking. However, since it is hard enough for most individuals to stop smoking, it is unlikely that they would simultaneously attempt to alter their eating habits.

Yet another problem associated with the use of smoking cessation products is one associated with diabetics. Many individuals are prone to having adverse varying levels of blood sugar. In such individuals, it would be helpful to provide a way to regulate the blood sugar.

What is needed, therefore, is a product that satisfies the psychological factors as well as the nicotine cravings experienced by an individual attempting smoking cessation. Further, a product that assists in regulating and/or balancing blood sugar is needed.

### SUMMARY OF THE INVENTION

The present invention relates to a composition and method for treating a condition responsive to nicotine therapy, particularly smoking and tobacco consumption cessation therapy and the reduction of nicotine cravings. The present invention is capable of introducing the nicotine into the patient's bloodstream quickly through an oral route, and is physically non-threatening and painless. Typically the composition of the present invention uses a candy base containing nicotine in the form of, for instance, a lollipop,

The method according to the present invention mimics the action of a smoker, (e.g., hand-to-mouth), and allows the smoker to control the amount of nicotine required to reduce a craving. It also allows the individual to consume nicotine in places where smoking is not permitted and to gradually eliminate smoking from specific environments until the individual no longer has the psychological associations of particular events or locations and tobacco consumption.

Absorption of nicotine through the buccal mucosa of the individual's oral cavity allows the nicotine to quickly enter an individual's bloodstream, giving an almost immediate relief from the craving and allowing the individual to determine when they have had enough and no longer feel the need to have a tobacco product. The lollipop may then be removed, or its rate of consumption decreased. The rate at which the nicotine is absorbed into the body can be varied by adjusting the rate the lollipop dissolves through the addition of hydrophobic or hydrophilic agents and by altering the concentration of the nicotine in the lollipop.

The present invention additionally relates to a method using nicotine replacement in conjunction with a smoking cessation program. This method further includes individual control and dosing of the nicotine replacement through the use of a selectively removable dosing mechanism.

This invention additionally includes the periodic substitution of the tobacco source and behavioral modification to reduce the craving and dependence on the tobacco product.

That the invention improves over the prior art and accomplishes the advantages described above will become apparent from the following detailed description of the exemplary embodiments and the claims that follow.

### DETAILED DESCRIPTION OF THE INVENTION

The present invention is directed to methods and compositions for use in the noninvasive, self-directed administration of nicotine as part of a program to stop the consumption of tobacco products. The present invention permits individual dosing and control over the administration of the nicotine, mimicking the action taken by a user of tobacco products and increasing the rate of successful cessation of cigarette consumption.

Transmucosal delivery of a drug is slower than administration by injection, nevertheless, the delay is offset by the noninvasive method of delivery which increases compliance. Moreover, given the number of people who try to quit smoking every year and fail, allowing individual dosing increases the success rate and allows a person to adjust the rate of nicotine consumption to meet individual needs.

In accordance with the present invention, the nicotine is dispersed in a candy base or form. The individual consumes as much of the candy as is necessary to offset the tobacco craving. The nicotine is released into the individual's mouth as the candy dissolves. A significant portion of the nicotine is absorbed through the mucosal tissues and rapidly enters the bloodstream. This method allows small amounts of the nicotine to be delivered to an individual continuously and administration of the nicotine may be stopped by the individual removing the candy when the craving is satisfied.

Further, through this method, the detrimental effect of smoke inhalation have been eliminated, even if the individual has difficulty overcoming the nicotine addiction. It is commonly understood that the primary cause of adverse health effects result from smoke inhalation and the combustion byproducts produced by cigarettes and other forms of tobacco, rather than the nicotine itself.

In contrast to the smoking cessation aids currently available, the current invention allows the individual to control the dosage. The method of administration of the present invention allows the individual to maintain a steady plasma concentration of the nicotine as opposed to wide swings in plasma concentrations typified by other methods of administration. For instance, use of currently available nicotine-containing gum may result in an overdose of nicotine. Since many smokers already chew gum, they are less likely to remove the gum after an appropriate dosage is administered, because of the pre-existing habit of chewing regular gum for long periods of time. The nicotine craving is quickly satiated when administered, for instance, in the form of a lollipop and then the lollipop is easily removed, preventing accidental overdose and allowing the user to easily decrease the amount of nicotine consumed in a systematic fashion.

A preferred method of administration includes use of a lollipop form. The use of a stick or other holder permits easy removal when the proper dosage has been received. The holder also facilitates intermittent administration of the nicotine to maintain the desired concentration of nicotine to decrease individual withdrawal symptoms. Further, use of a lollipop aids the smoker psychologically, as the insertion and removal of the lollipop closely mimics insertion and removal of a cigarette, (e.g., hand-to-mouth), thereby satisfying a psychological portion of the habit.

The consumption of the candy can be altered as necessary to increase or decrease the amount of nicotine in the system, (e.g. the body), by the length of time the candy is held in the mouth. Varying the concentration of the drug in relation to the candy base will also affect the amount of nicotine that enters the system.

Additives such as flavorings, sweeteners, flavor enhancers, releasing agents or buffers may be added to make the lollipop more palatable. Any number of flavorings may be combined in order to produce the specific characteristics desired. Conventional sweeteners such as sucrose may be utilized, or carbohydrates suitable for use with diabetics such as sorbitol. Other sweeteners such as aspartames or nutrasweet can be used as part of the dietary control needed by those withdrawing from nicotine consumption. Also, natural sweeteners, such as stevia, an herb in the chrysanthemum family, may be used.

A preferred composition according to the present invention typically includes the following ingredients:

Candy base 8.47 g

Stevia 0.0055 cc

Chromium picolinate 0.0055 ml

Nicotine salycilate 0.5-10 mg

Typically, a candy base is melted at a temperature of about 90-100° C., along with an appropriate sweetener, to which a flavoring or coloring may be added. The mixture is allowed to cool to between 70-90° C., at which time nicotine salicylate is typically added to the mixture. The resulting product may be stirred or agitated until smooth and homogenous, at which point it may be poured into an appropriate mold.

In the formulations or compositions according to the present invention, low doses of nicotine are included, typically in an amount less than 10 mg, and more typically 2-4 mg. Additionally, the resulting product may be buffered to aid in buccal absorption of the nicotine. Preferred buffering components include, but are not limited to, sodium carbonate, sodium phosphate, calcium carbonate, magnesium hydroxide, magnesium carbonate, aluminum hydroxide or other substances known to those skilled in the art.

According to a pertinent aspect of the present invention, that has heretofore not been contemplated, chromium picolinate is added to the composition to effectively combat the overwhelming, (and sometimes seemingly unsurmountable), problem associated with smoking cessation—weight gain. The chromium picolinate impressively functions as an appetite suppressant to counter balance the decrease in the appetite suppressant activity of the nicotine with the decreasing nicotine dosage. Though not wishing to be bound by any theory, it is thought that chromium picolinate acts to regulate the blood sugar through increasing the number of insulin receptors in the body. Chromium picolinate can also lower total cholesterol and triglycerides and increase HDL levels in nondiabetic patients. Typically, chromium picolinate is added to the composition in an amount of about 100-400 mcg, preferably about 200 mcg.

### EXAMPLES

The above disclosure generally describes the present invention. A more complete understanding can be obtained by reference to the following examples. These examples are described solely for purposes of illustration and are not intended to limit the scope of the invention. Although specific terms have been employed herein, such terms are intended in a descriptive sense and not for purposes of limitations.

### Example 1: Preparation of Lollipop

A candy base for the nicotine lollipops within the scope of the present invention may be prepared utilizing candy preparation formulas and techniques which are known in the art.

At the same time, a solution containing the nicotine is prepared at the desired concentration.

A total of 305 grams of candy base and 2 cc of stevia were melted at a temperature of about 95° C. Additionally, 2 ml of chromium picolinate in 7.2 mg/2 cc ethyl alcohol, (about 200 mcg chromium picolinate per lollipop), was added to the mixture and it was allowed to cool to about 90° C., at which time 2 ml of nicotine salicylate at a concentration of 36 mg/ml in ethyl alcohol was added and mixed until smooth and homogenous. The resulting mixture was allowed to cool to about 75° C., at which time the mixture was poured into a mold of 36 lollipops and holders were placed into the mixture and allowed to harden for 6 to 8 hours.

### Example 2: Case Study

Patient A is an adult female who is 45 years of age. She has smoked 1.5 packs per day since she was 23 years old. She has tried to stop smoking several times and has managed to quit smoking for as long as 2 years. However, she usually lapses more quickly. She has tried hypnoticsuggestion, the nicotine patch, and nicotine gum, as well as quitting cold turkey. Patient A smokes more during times of stress or boredom, in conjunction with alcohol consumption and when she socializes with other smokers. When she is stuck in traffic, she smokes more. She also has frequent smoke breaks during the day and smokes on her way home from work. After numerous attempts to stop smoking, she tried the nicotine lollipop according to the present invention's method and composition.

After consulting her physician, Patient A filled a prescription for the 4 mg lollipop. She initially began using the 4 mg lollipop using the method of substitution as described herein. The method as described herein does not require the patient to discontinue using cigarettes at the most stressful times, but instead calls for a plan, commitment and implementation of substitution of individual cigarettes. The first week, she used the lollipop for two of her daily smoke breaks at work. During the second week, Patient A substituted the lollipop for smoking in the automobile. With each substitution, Patient A was committed to discontinuing that practice. The third week, the lollipop was substituted for cigarettes after meals. The fourth week's commitment and implementation was substitution of the cigarette with a cup of coffee. Discontinuing smoking while talking on the telephone was the objective of the fifth week. With each consecutive week, Patient A successfully substituted the lollipop for various cigarettes, and was able to successfully discontinue smoking after twelve weeks.

The terms and expressions which have been employed herein are used as terms of description and not of limitation, and there is no intention, in the use of such terms and expressions, of excluding any equivalents of the features shown and described or portions thereof. Having thus described the invention in detail, it should be apparent that various modifications can be made in the present invention without departing from the spirit and scope of the following claims.

I claim:

1. A method for satisfying the craving for cigarettes and thereby resulting in smoking cessation by inserting a nicotine containing lollipop into an oral cavity of an individual for absorption into a buccal mucosa, said lollipop gradually releasing a measured dose of nicotine, said method comprising a stepwise substitution of the lollipop for a cigarette until smoking ceases.

2. A composition for smoking cessation comprising a candy base, a sweetener, chromium picolinate, and nicotine.

3. The composition of claim 2, wherein said sweetener is a natural sweetener.

4. The composition of claim 2, wherein said composition comprises less than about 10 mg of said nicotine.

5. The composition of claim 2, wherein said composition comprises 2-4 mg of said nicotine.

6. The composition of claim 2 wherein said composition comprises 200 mcg of said chromium picolinate.
